(19) United States
(12) Patent Application Publication
(10) Pub. No.: US 2022/0249259 A1
(43) Pub. Date: Aug. 11, 2022

(54) PROSTHETIC ADAPTER

(71) Applicant: LIFEWORK ADAPTER LLC, AUSTIN, TX (US)

(72) Inventor: MARSCHELL SAADON, Austin, TX (US)

(21) Appl. No.: 17/630,462

(22) PCT Filed: Jul. 27, 2020
(86) PCT No.: PCT/US2020/043771

§ 371 Date: Jan. 26, 2022

Related U.S. Application Data
(60) Provisional application No. 62/879,456, filed on Jul. 27, 2019.
(60) Provisional application No. 63/007,216, filed on Apr. 8, 2020.

(51) Int. Cl. A61F 2/58; B25B 5/06

(52) U.S. Cl. CPC A61F 2/588; B25B 5/06

(57) ABSTRACT

A prosthetic device adaptor includes a tool interface, a coupling connected to the tool interface, and a prosthetic interface connected to the coupling. The prosthetic interface can connect with a Hosmer-style hook. The tool interface can be secured to a tool. The tool interface can include a strap, a circular clamp, a c-clamp, or a threaded connector. The coupling can have two parts that separate or detach from one another. The coupling can permit rotation around an axis.

### CROSS-REFERENCE TO RELATED APPLICATION(S)

This application is a 371 of International Application of PCT/US2020/043771, filed Jul. 27, 2020, which claims benefit of U.S. Provisional Application No. 62/879,456, filed Jul. 27, 2019, and which claims benefit of U.S. Provisional Application No. 63/007,216, filed Apr. 8, 2020, each of which is incorporated herein by reference in its entirety.

### BACKGROUND

Globally, hundreds of thousands of people lose limbs every year. As a result, they lose access to the jobs for which they were trained and lose the ability to enjoy hobbies they love. While such injuries represent significant monetary losses to employers, insurance companies, and the economy, the loss to the individual is immeasurable. They lose access to the many activities others often take for granted.

Yet, for many decades, relatively few prosthetic options have been available for amputees. Typical hook or clamp-like devices are available but have limited usability. Despite their common use, they do not interface well with power tools and gardening tools.

More recently, attempts have been made to develop robotic arm prosthetics. However, such robotic prosthetics are expensive to purchase, and training is costly. Moreover, such robotic prosthetics are currently too slow to use with power tools.

### BRIEF DESCRIPTION OF THE DRAWINGS

The present disclosure may be better understood, and its numerous features and advantages made apparent to those skilled in the art by referencing the accompanying drawings.

FIG. 1 includes an illustration of an example tool connected to a prosthetic adapter.

FIG. 2 includes an example tool attached to prosthetic adapter.

FIG. 3 includes an exploded view of an example prosthetic adapter.

FIG. 4 includes an illustration of a further view of an example prosthetic adapter.

FIG. 5 includes an illustration of an example tool interface.

FIG. 6 includes an example of a strap.

FIG. 7 includes an example of a coupling.

FIG. 8 is an illustration of an exploded view of an example coupling.

FIG. 9 includes an illustration of an example prosthetic platform.

FIG. 10 includes an illustration of an example prosthetic interface.

FIG. 11 includes an illustration of an example prosthetic interface with couplings.

FIG. 12 includes an illustration of an example prosthetic adapter.

FIG. 13 and FIG. 14 include illustrations of an example prosthetic interface.

FIG. 15 and FIG. 16 include illustrations of an example prosthetic platform.

FIG. 17 and FIG. 18 including illustration of an example tool interface.

FIG. 19 includes an illustration of an example portion of the tool interface.

FIG. 20 and FIG. 21 include illustrations of an example tool interface attached to a power saw.

FIG. 22 includes an illustration of an example tool interface.

FIG. 23 and FIG. 24 include illustrations of an example prosthetic adapter attached to a chainsaw.

FIG. 25 and FIG. 26 include illustrations of an example prosthetic adapter attached to a weed eater.

FIG. 27 includes an illustration of an example tool interface attached to a grinder.

FIG. 28 and FIG. 29 include illustrations of an example tool interface.

FIG. 30 includes an illustration of an example prosthetic adapter attached to a circular saw.

The use of the same reference symbols in different drawings indicates similar or identical items.

### DETAILED DESCRIPTION

In an example, a prosthetic adapter can connect to various tools and provides an interface for interaction with conventional prosthetics for upper-body limb amputees. For example, the most common conventional prosthetic for an upper-body limb amputee is the prosthetic Hosmer-like hook. In an example, a prosthetic adapter includes a tool interface coupled to a coupling, which is coupled to a prosthetic interface. For example, the prosthetic interface interfaces with Hosmer-like hooks.

In an example, the prosthetic adapter includes a prosthetic interface coupled with a tool interface. The prosthetic interface can operably interact with a prosthetic, such as an upper body prosthetic. In particular, the upper body prosthetic can be a prosthetic hook, such as a Hosmer-like prosthetic hook, for example, a Hosmer 5 or Hosmer 7 prosthetic hook. The tool interface can have various configurations designed to interact with different tools. For example, the tool interface can be configured to couple to a tool using a strap. In another example, the tool interface can be configured to couple to a tool using a circular clamp. In another example, the prosthetic interface can be designed to couple to a tool using a c-clamp like interface. In a further example, the tool interface can directly integrate with the frame or cover of a tool, for example, using a threaded interface or being welded to the frame or cover. In particular, the tool interface can be configured to couple to power tools such as drills, power drills, circular saws, reciprocating saws, jigsaws, chainsaws, grinders, sanders, or the like. In another example, the tool interface can be configured interface with gardening tools, such as weed eaters, edgers, hedge clippers, lawnmowers, shovels, hoes, rakes, tree trimmers, shears, clippers, or the like.

The prosthetic interface is coupled to the tool interface. For example, the prosthetic interface can be fixedly coupled to the tool interface. In another example, the prosthetic interface can be coupled to the tool interface using a detachable coupling, such as a quick release coupling. In an example, the coupling between the prosthetic interface and the tool interface can be fixed, not allowing for rotation or movement. In another example, the coupling between the prosthetic interface and the tool interface can allow rotation around a single axis, such as an axis defined through the coupling from the prosthetic interface to the tool interface. In another example, the coupling between the prosthetic interface and the tool interface can permit two-axis movement.

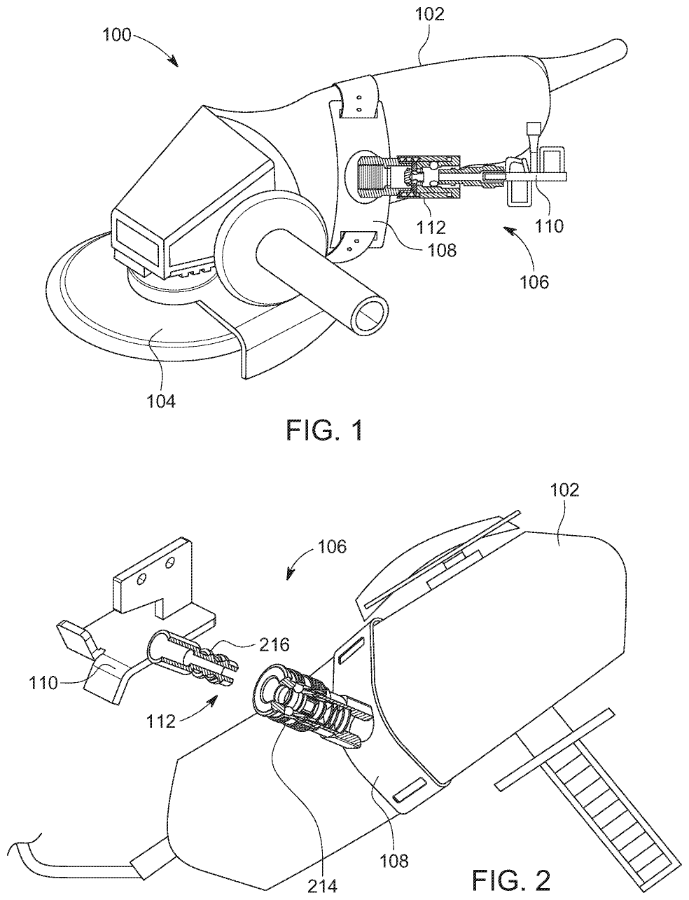

For example, FIG. 1 includes an illustration of an adapted tool 100 that includes a tool body 102 and active portion 104. The illustrated tool includes a grinder. Alternatively, the tool can be a drill, power drill, reciprocating saw, circular saw, jigsaw, or chainsaw, among other tools. A prosthetic adapter 106 is coupled with the tool body 102. For example, the prosthetic adapter 106 includes a tool interface 108 that attaches to the tool body 102. The prosthetic adapter 106 further includes a coupling 112 connected to the tool interface 108 and a prosthetic interface 110. In particular, the coupling 112 rotates around an axis.

For example, as illustrated in FIG. 2, the coupling 112 can be formed of two parts, a male portion 216 and a female portion 214. The male portion 216 and the female portion 214 can connect in a manner that allows the male portion 216 to rotate around an axis defined by the male and female portions. As illustrated, the portions 214 and 216 can be detached and reattached. Alternatively, the portions 214 and 216 can be fixed so that they are rotatable, but not detachable.

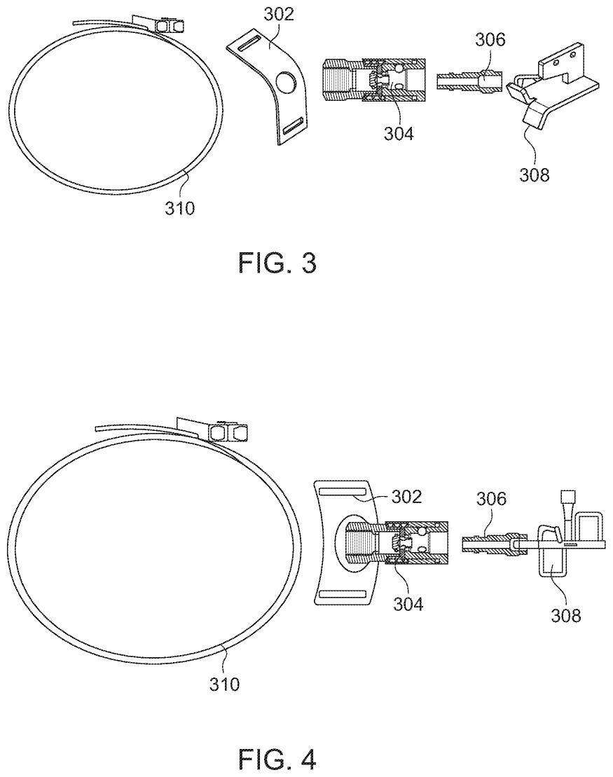

FIG. 3 illustrates an exploded view of the parts of an example prosthetic adapter. For example, a tool interface plate 302 can be attached to a strap 310 that couples with a tool. A female portion 304 of the coupling can be attached to the tool interface plate 302. The female portion 304 of the coupling can interact with a male portion 306 the coupling that attaches to the prosthetic interface 308.

While FIG. 3 illustrates the female portion of the coupling 304 being associated with the tool interface plate 302 and the male portion 306 of the coupling being attached to the prosthetic interface 308, the female portion 304, alternatively, can be attached to the prosthetic adapter interface 308 and the male portion 306 can be attached to the tool interface plate 302.

FIG. 4 further illustrates the parts. For example, the tool interface plate 302 can be fixedly attached to a coupling portion 304 of the coupling. In an example, the coupling portion 304 can be welded to the interface plate 302. Alternatively, the portion 304 can be adhered to the faceplate 302 or fixed with a fastener, such as a rivet or screw. As illustrated in FIG. 4, the coupling portion 306 is connected to or fixedly coupled with the prosthetic interface 308. In particular, the portion 306 of the coupling can be inserted into the portion 304 of the coupling, attaching the prosthetic interface 308 to the tool interface plate 302 via the coupling in a rotatable manner.

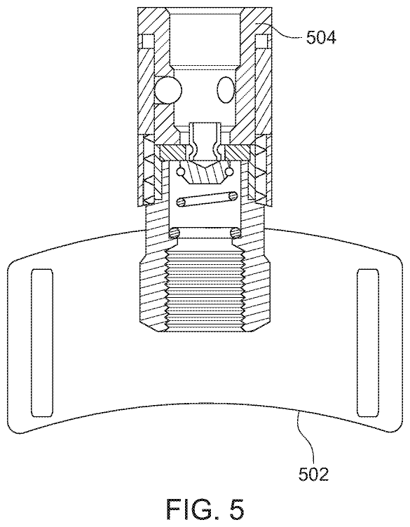

FIG. 5 includes further illustration of the interface to the tool. For example, the tool interface plate 502 can be coupled to a portion of a coupler 504 that creates a rotatable coupling. The tool interface plate 502 can be formed of a metal plate, plastic, or natural products such as leather. For example, the tool interface plate 502 can be a rigid material such as a metal or a rigid plastic. In another example, the tool interface plate 302 can be flexible such as a thin sheet of metal, a rubber, or a natural material, such as leather. In an example, the portion 504 of the coupling can be formed of a rigid material, such as metal or plastic.

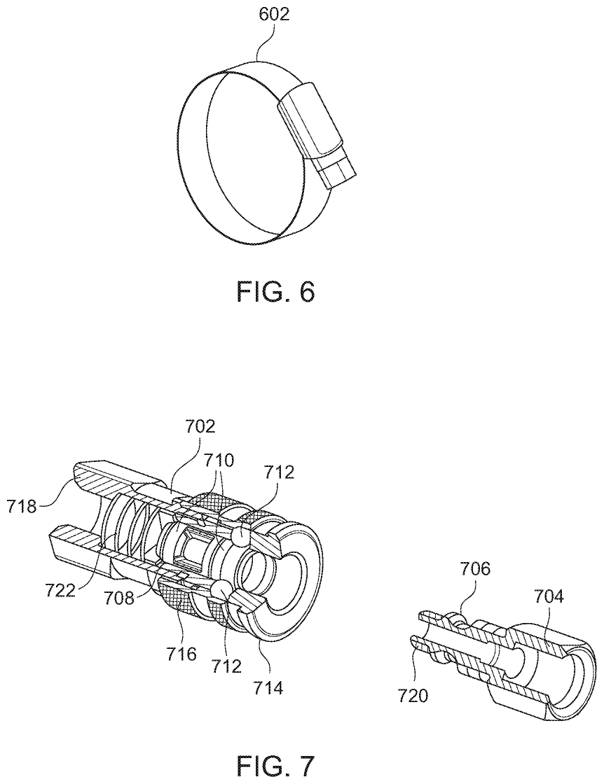

As illustrated in FIG. 6, a clamp 602 can be formed of a strap that can be adjusted to secure the tool interface to the tool. In an example, the strap can include a leather or rubber strap or a pipe coupling. Optionally, the strap can include a quick disconnect.

In an example, the portions of the coupling can be formed of a rigid material, such as metal or plastic. For example, as illustrated in FIG. 7, a female portion 702 of the coupling includes a mounting base 718 mechanically fastened to a tool interface plate on a tool. A male inlet guide 714 can act as a ball bearing housing and slip ring stop that receives the male contact connection shaft. The male inlet guide 714 can be coupled with the mounting base 718, for example, using a threaded connection. An external slip ring and locking mechanism 716 can lock and unlock ball bearings 712. The female portion 702 can further include magnetic mounting surfaces and spring guide 708 or 722 or a spring-loaded retention ring and internal magnetic base 710.

The male portion 704 can include a male shaft protrusion ball bearing interface 706 and optionally a male magnetic passive secondary locking point 720. The male portion 704 can be hollow or can be solid.

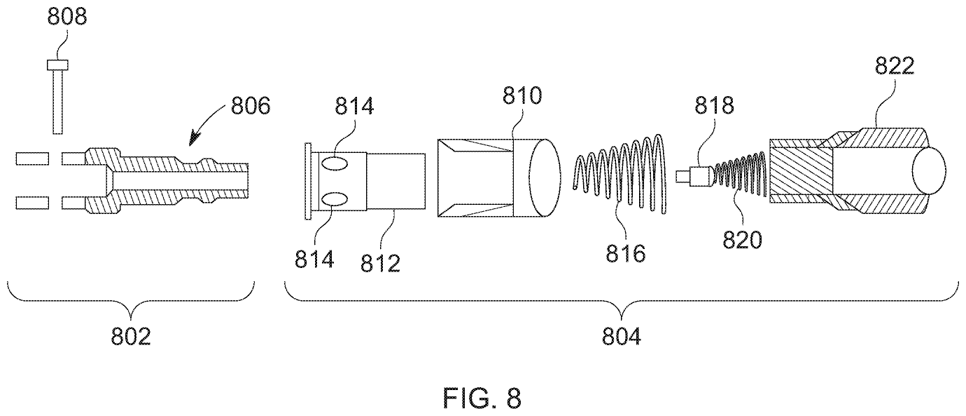

FIG. 8 illustrates another embodiment of the rotatable coupling between the tool interface and a prosthetic interface. For example, the male portion 802 of the coupling includes a shaft contoured to included ball bearing capture valley 806. Further, the system can include a fasting screw 808 to fasten the male portion 802 to the prosthetic adapter interface.

The female portion 804 can include a lower housing 822. The lower housing is optionally threaded to connect with the ball bearing housing 812. The ball bearing housing 812 includes ball bearings 814. A locking sleeve compression spring 816 and ball bearing locking sleeve 810 can slide over the bearing housing 812 when it is secured to the lower housing 822. Optionally, the system can include a magnetic mount 818 and magnetic locking spring 820 that extends internally with the internal ball bearing housing 812 when the housing is secured to the lower housing 822.

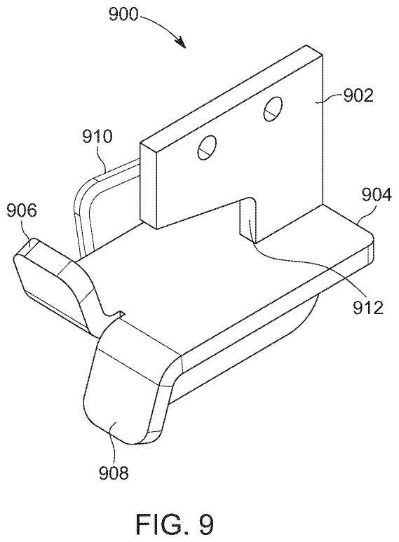

FIG. 9 illustrates an example prosthetic interface 900. The interface 900 includes a post plate 902 secured to a grip plate 904. The post plate 902 includes a concave surface 912. The grip plate 904 extends to either side of the post plate 904 and out from the concave surface 912. At a distal end of the grip plate 904 distal from the concave surface 912, a lip 906 can be formed. In an example, the lip extends in a first direction. Optionally, a second lip 908 extends in an opposite direction.

Optionally, a loop 910 is formed on an edge of the grip plate 904. In particular, the loop 910 is formed on opposite side of the grip plate 914 from where a hook is to initially engage. When engaged, the hook slides across the grip plate 904 with a tip entering the loop 910 and the central portions of the hook engaging the concave surface 912 of the post plate 902. In a particular example, the post plate 902 is formed of a metal sheet material. Alternatively, the post plate can be formed of a rod or take various forms. In an example, the grip plate 904 is formed of sheet metal or plastic.

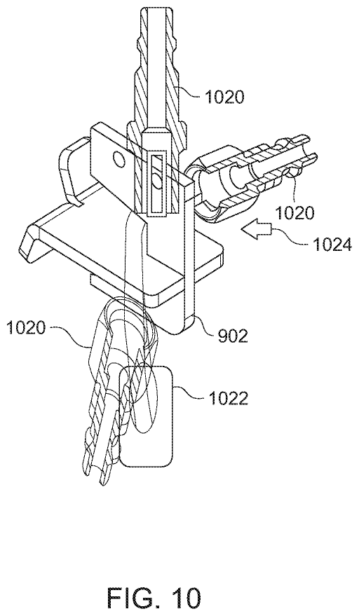

FIG. 10 includes a further illustration in which the prosthetic interface 902 is connected to a portion of the coupling 1020. As illustrated, the coupling 1020 can be connected at various positions to the post plate 902 or alternatively to the grip plate 904. In particular, a prosthetic 1022 including the Hosmer-like hook 1024 can slide such that the hook portions of the Hosmer-like hook attaches to either surface of the grip plate 904 and are secured against the concave surface of the post plate 902 and optionally slide into the loop 910.

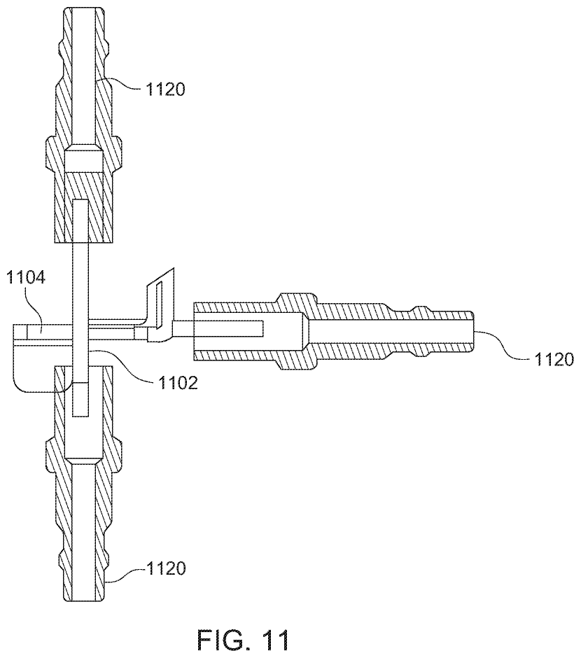

FIG. 11 includes a further illustration as a side view illustrating the coupling portions 1120 connected to either a post plate 1102 of the prosthetic adapter or the grip plate 1104 of the prosthetic adapter.

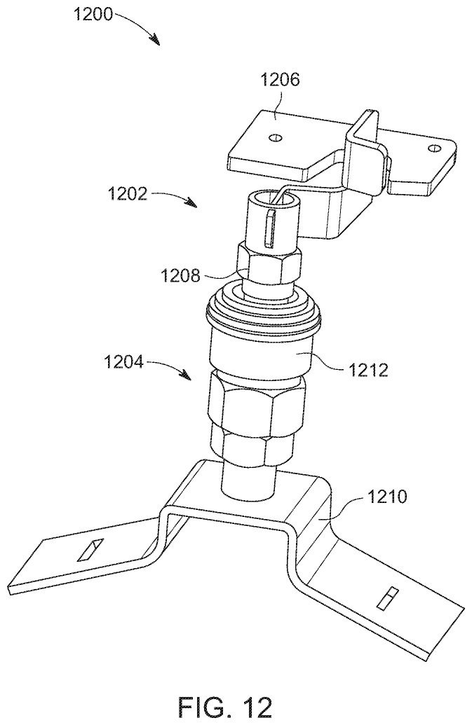

FIG. 12 includes a further illustration of a prosthetic adapter 1200 including a prosthetic interface 1202 and a tool interface 1204. The prosthetic interface 1202 includes a platform 1206 for connecting with a prosthetic device, such as an upper body prosthetic device, for example, including a Hosmer-like hook. The prosthetic interface 1202 further includes a portion 1208 of a coupling. As illustrated, the portion 1208 includes a male end of the quick connect coupling.

In an example, the tool interface 1204 includes a portion 1210 configured to connect to the tool and a portion 1212 of the coupling. As illustrated, the portion 1212 of the coupling is a female portion of the quick connect coupling for receiving the male portion 1208 attached to the prosthetic interface 1202.

The portion 1210, which is to connect to the tool, can take various configurations. As illustrated, the portion 1210 of the tool interface 1204 is configured to receive a strap that connects the portion 1210 to a tool. Alternatively, the portion 1210 can include a circular clamp, a c-clamp, a threaded connector, or other connectors to interact with a specific tool.

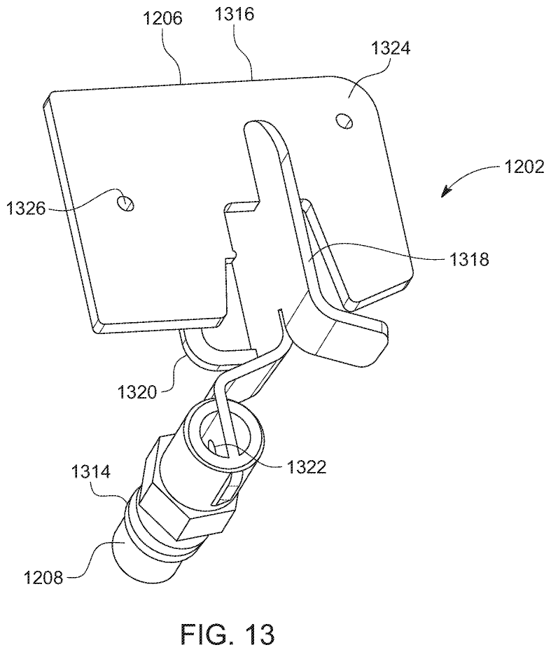

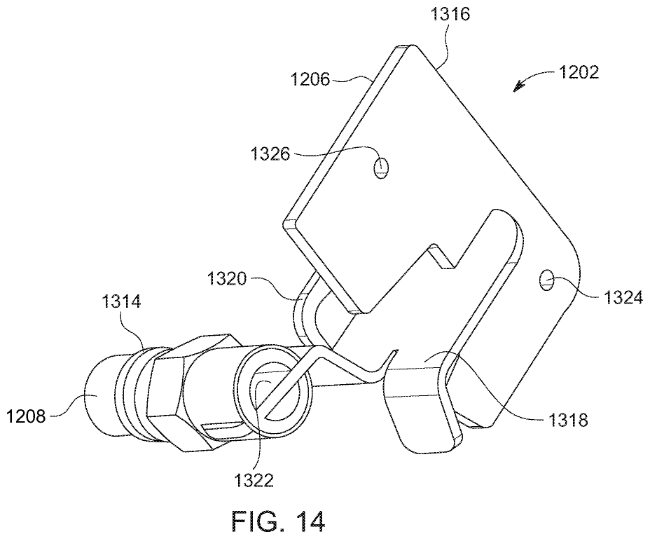

FIG. 13 and FIG. 14 include illustrations of the example prosthetic interface 1202. The prosthetic interface 1202 includes a platform 1206 to interact with a prosthetic device and includes the portion 1208 of the coupling for connecting with the tool interface.

The platform includes a post plate 1316, a grip plate 1318, and optionally, a loop 1320. The prosthetic device, such as a prosthetic hook, can engage the grip plate 1318 and optionally, the loop 1320. When positioned in place, the post plate 1316 further secures top and bottom surfaces of the prosthetic device.

As illustrated, the portion 1208 of the coupling is a male portion of a quick connect coupling. Alternatively, the portion 1208 can be a female portion of coupling. The male portion of the quick connect coupling includes a contour 1314 configured to engage the female portion of the quick connect coupling, for example, including a ridge to engage bearings of the female portion of the quick connect coupling.

The portion 1208 of the coupling is illustrated as being connected to the platform 1206 at position 1322. The portion 1208 is further illustrated as being directed parallel to the grip plate 1318 with the coupling directed in the direction of the loop 1320. The portion 1208 can be connected with a screw, bolt, pin, or welding to the platform 1206. Alternatively, the portion 1208 can be connected at different angles at position 1322.

In a further example, the portion 1208 of the coupling can be connected at other positions on the platform, such as position 1324 or 1326 at various angles. For example, the coupling portion 1208 can be connected at position 1324 or at position 1326 and extend parallel to the post plate 1316 at various angles. Depending upon the nature of the tool to which the prosthetic adapter is to be connected, a desired angle at which the prosthetic engages the platform can be different. As such, the portion 1208 of the coupling can be attached to the platform 1206 at angle and position desirable for interacting with a particular tool.

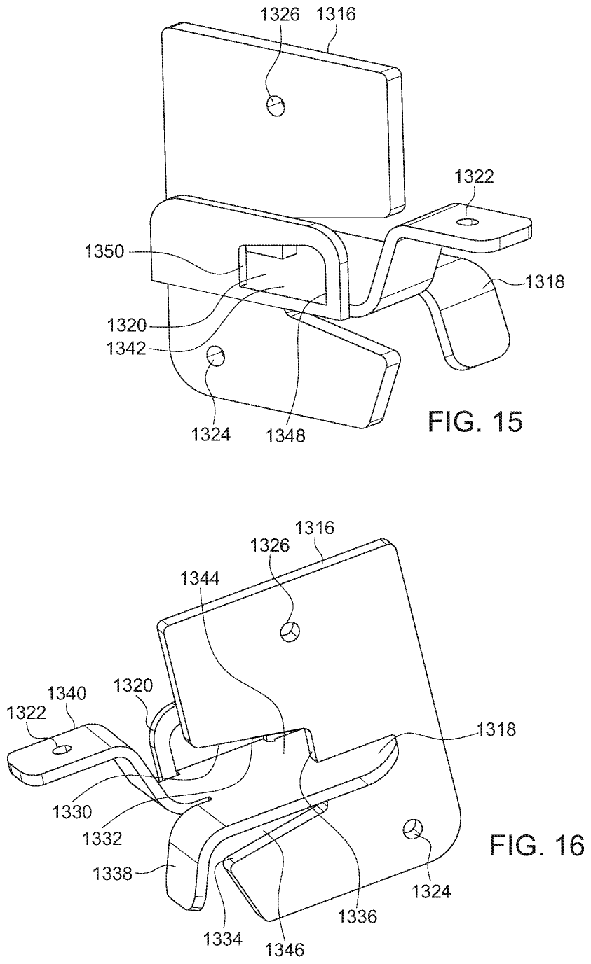

FIG. 15 and FIG. 16 include illustrations of the platform 1206. A post plate 1316 is connected to a grip plate 1318. For example, the post plate 1316 can be coupled to the grip plate 1318 in an approximately perpendicular fashion. The post plate 1316 can be formed of material such as sheet material, such as a sheet metal. Example metals include stainless steel, steel, aluminum, and titanium, among others. In another example, the post plate 1316 can be formed of a polymeric material. Alternatively, the post plate 1316 can be formed of a rod or other material Similarly, the grip plate 1318 can be formed of sheet material similar to those described above.

The post plate 1316 can include a concave surface 1330 that defines an upper ridge 1332 extending over an upper surface of the grip plate 1318 and a lower ridge 1334 extending under a lower surface of the grip plate 1318. The grip plate 1318 can extend at 1336 from the concave surface 1330 of the post plate 1316. One or more lips 1338 or 1340 can be defined at an end of the grip plate 1318 distal from the concave surface 1330 of the post plate 1316. For example, a lip 1340 can extend out of the plane defined by the grip plate 1318 in an upward direction. Another lip 1338 can extend in a different direction, such as a downward direction out of the plane defined by the grip plate 1318. The grip plate 1318 can further define a loop 1320 at an edge of the grip plate 1318. For example, the loop 1320 can define an opening 1342. The loop 1320 can be located at an edge of the grip plate 1318 distal from the post plate 1316 and parallel to the post plate 1316.

In practice, when engaging a Hosmer-like hook, such as a Hosmer 5 or Hosmer 7 prosthetic hook, the hook can engage the adapter from the side opposite the loop 1320. For example, an upper portion or finger of the hook can engage a top surface of the grip plate 1318 extending into the cavity 1344 defined between an upper ridge 1332 of the concave surface 1330 and an upper surface of the grip plate 1318. Another finger of the hook can enter the cavity 1346 defined between a lower ridge 1334 of the post plate 1316 and the lower surface of the grip plate 1318. An end of a finger of the hook can extend around the post plate 1316 and engage the opening 1342 of the loop 1320.

In particular, the upper ridge 1332 of the concave surface 1330 can engage a rearward portion of the concave surface at angles such as in a range of 90° to 130°, a range of 95° to 120° or even a range of 95° to 110°. Similarly, the lower ridge 2112 can engage a rearward surface of the concave surface 2114 at such angles.

An opening to the cavity 1344 defined between the lip 1340 and a closest point of the post plate 1316 can have a width in a range of 0.25 inches to 0.35 inches, such as a width in a range of 0.26 inches to 0.3 inches or a range of 0.275 inches to 0.285 inches. An opening defined into cavity 1344 between the lip 1340 and the closest surface of post plate 1316 can have a width in a range of 0.28 inches to 0.35 inches, such as width in a range of 0.3 inches to 0.35 inches or even 0.325 inches to 0.34 inches.

The loop 1320 can define the opening 1342 to receive a tip of a finger of the Hosmer-like prosthetic hook. In particular, the opening 1342 can be tapered, being larger at a forward surface 1348 and shorter at a rearward surface 1350. For example, the opening at the forward surface 1348 can be in a range of 0.29 inches to 0.35 inches, such as a range of 0.3 inches to 0.33 inches or even a range of 0.3 inches to 0.32 inches. The rear surface 1350 of the opening 1342 can have a height in a range of 0.21 inches to 0.25 inches, such as a range of 0.22 inches to 0.24 inches or even a range of 0.23 inches to 0.24 inches.

In practice, as the Hosmer-like hook engages the adapter, it wraps around the post plate 1316 and has a tip of the upper finger enter into the opening 1342, the hook is drawn rearward and is pinched both by the height between the post plate 1316 and the grip plate 1318, as well as being secured and pinched by the restricted height of the opening 1342. While FIG. 15 and FIG. 16 illustrate a prosthetic platform configured for a left hand prosthetic hook, a mirror of the parts can be used to form a platform configured for a right hand prosthetic hook.

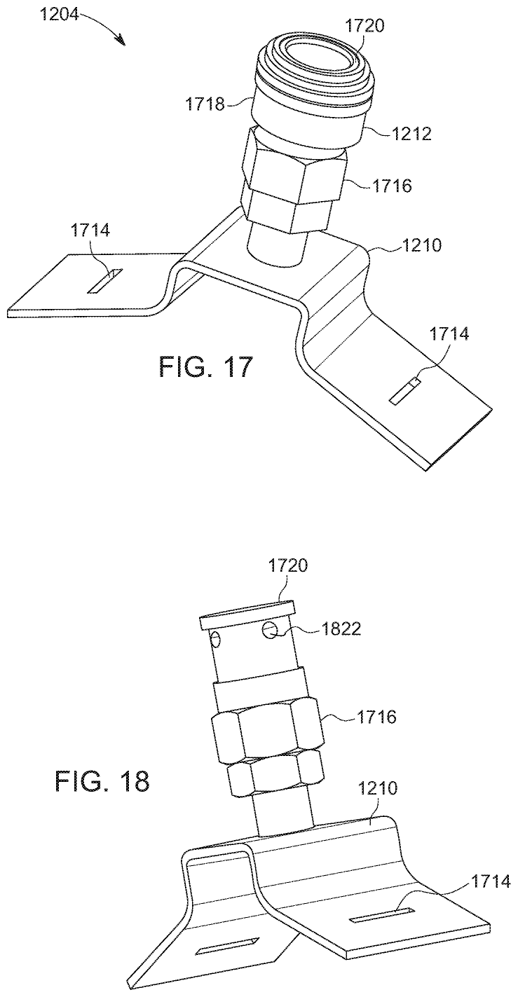

FIG. 17 and FIG. 18 include illustrations of an example tool interface 1204 including a portion 1210 to connect with the tool and a portion 1212 for connecting with the prosthetic interface 1202. In the illustrated example, the portion 1212 can be a female portion of a coupling, such as that illustrated in FIG. 7. The slip ring 1718 can slide along the male inlet guide 1720 to permit insertion of the male portion 1208 of the coupling. Movement of the slip ring 1718 allows the bearings 1822 to move, allowing the male portion to enter and be locked into place within the male inlet guide 1720.

The portion 1212 of the coupling can be secured to the portion 1210 to interface with the tool by a post 1716. For example, the post 1716 can be secured to the portion 1210 with a bolt. In another example, the post 1716 can be secured to the portion 1210 using welds. In the illustrated example, the post 1716 is secured to the portion 1212 of the coupling using a threaded connection.

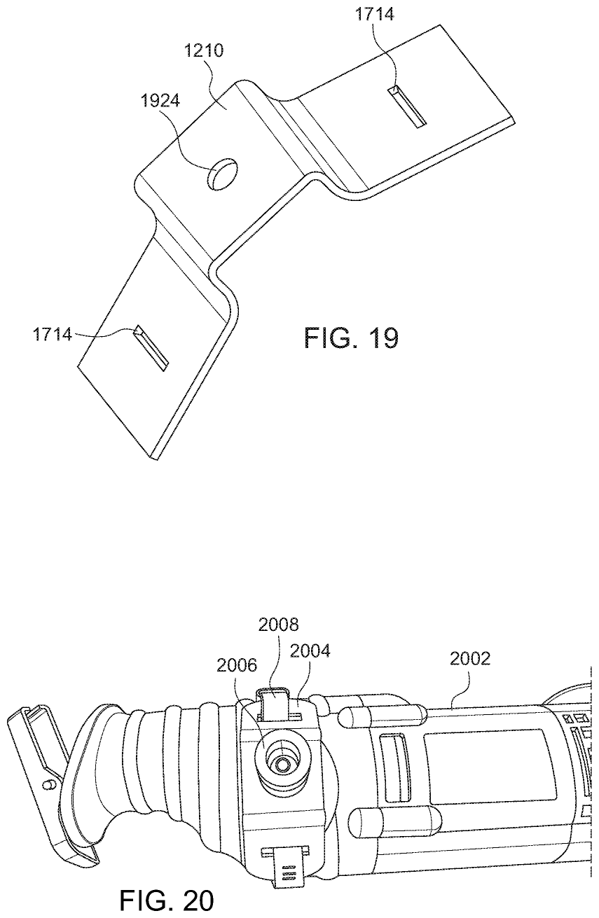

As illustrated, the portion 1210 is configured to receive a strap through slots 1714. For example, as illustrated in FIG. 19, the portion 1210 includes slots 1714 to engage a strap surrounding a tool. Further, the portion 1210 can include an opening 1924 to engage the post 1716. For example, the portion 1210 can be connected to the post by inserting a bolt through the opening 1924 and threadedly securing the post 1716 to the portion 1210.

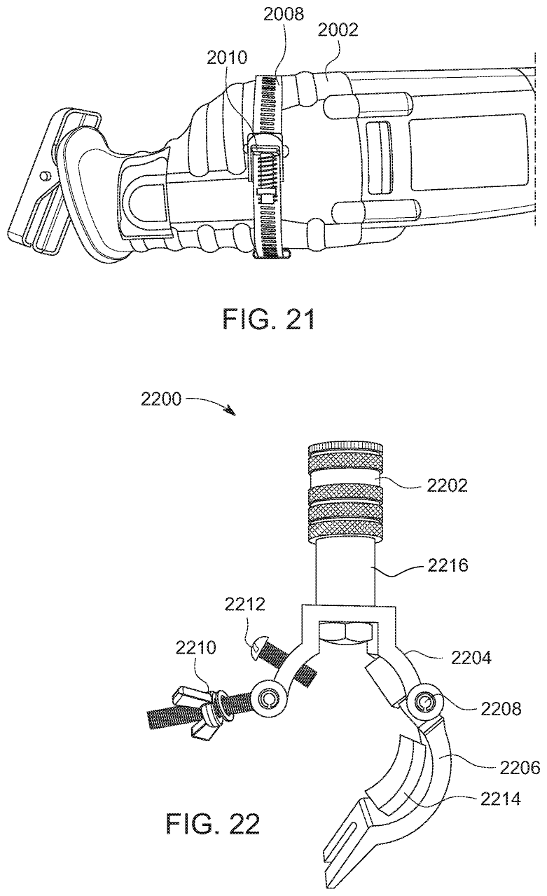

FIG. 20 and FIG. 21 include illustrations of a tool interface, such as the tool interface 1204 secured to a tool. As illustrated, the tool 2002 is a reciprocating saw coupled to tool interface 2006 using a strap 2008 and a tool portion 2004 of the tool interface 2006. As illustrated in FIG. 21, the strap 2008 can include slits that are engaged by a fitting 2010 that permits the strip 2008 to be tightened by rotating a threaded pin of the fitting 2010. Optionally, the threaded fitting 2010 can be a quick release fitting.

FIG. 22 illustrates an alternative embodiment of a tool interface 2200. As illustrated, the tool interface 2200 includes a coupling portion 2202, such as a quick connect coupling. In addition, the tool interface 2200 includes a tool interface portion 2204 in the form of a circular clamp. A lower portion 2206 of the circular clamp can rotate around a pivot 2208 to enclose a cylindrical interface of the tool. The lower portion 2206 can engage a connector 2210 that can, for example, screw into place to secure lower portion 2206 to the interface portion 2204. In addition, the interface portion 2204 can include a set screw 2212 to further secure the cylindrical tool interface within the tool portion 2204. Optionally, elastomeric material 2214 can be disposed around the inner circumference of the tool interface 2204. In the illustrated example, the portion 2202 of the coupling is coupled to the tool interface portion 2204 using a bolt post 2216.

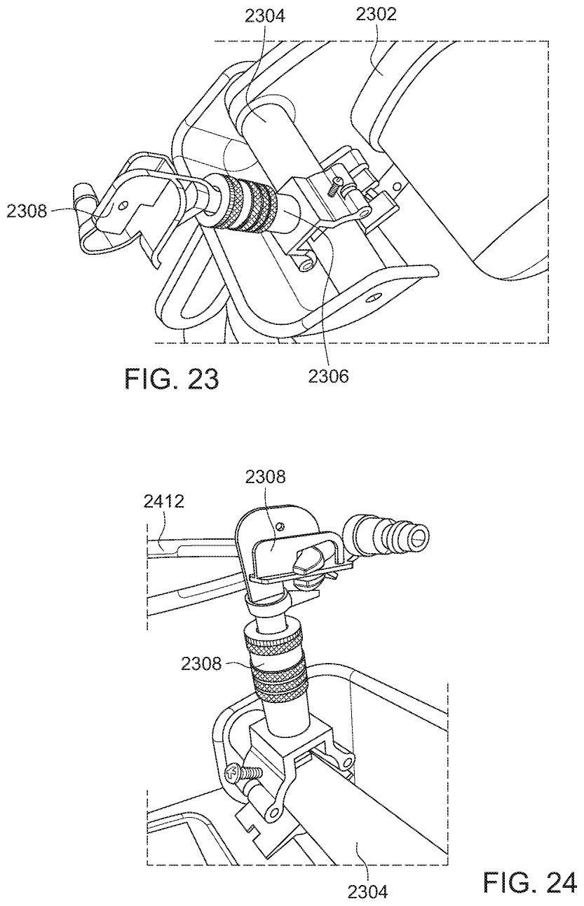

Such a circular connector 2200 can coupled to various post-like tool interfaces. In an example, such a tool interface 2200 can couple to shovels, hoes, rakes, or various handles of power tools. For example, such a connector can connect to a handle of a chainsaw, as illustrated in FIG. 23 and FIG. 24. In particular, the chainsaw 2302 includes a circular handle 2304 to which the interface 2306 is connected. A prosthetic interface 2308 is connected to the tool interface 2306. As further illustrated in FIG. 24, a hook device, such as a Hosmer-like prosthetic hook 2412 can engage the prosthetic interface to 2308, which is connected to the tool interface 2306.

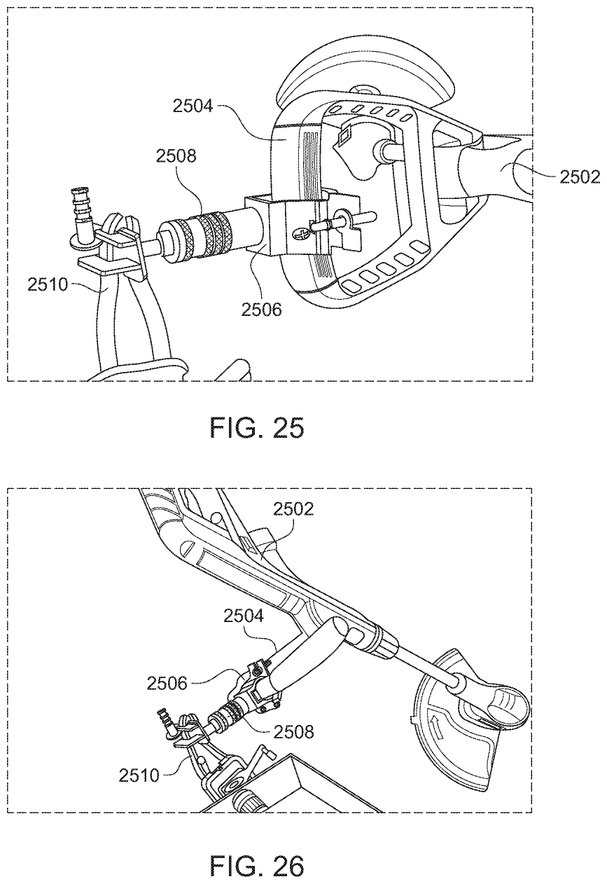

In another example illustrated in FIG. 25, a weed eater 2502 includes a circular handle 2504. The tool interface 2506 can engage the circular handle 2504 of the weed eater 2502. A prosthetic interface 2508 coupled to the tool interface 2506 can engage a prosthetic hook 2510, as illustrated in FIG. 26.

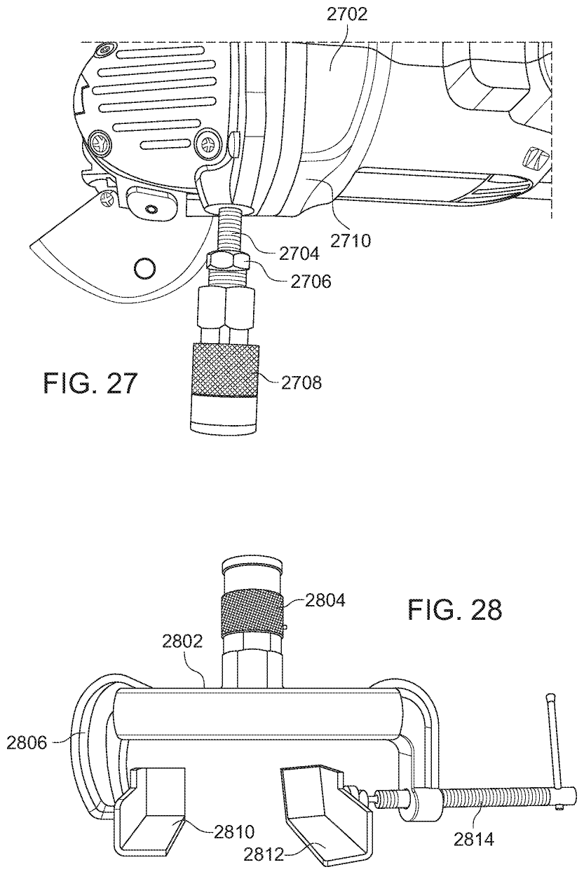

In a further example, the tool interface can directly couple with the frame of a tool. For example, as illustrated in FIG. 27, a grinder 2702 includes a threaded interface 2704 integrated with the frame 2710 of the tool. The tool interface 2706 can be directly connected to the frame 2710 of the tool 2702. The threaded connection 2706 directly connects to the coupling portion 2708 to the tool 2702.

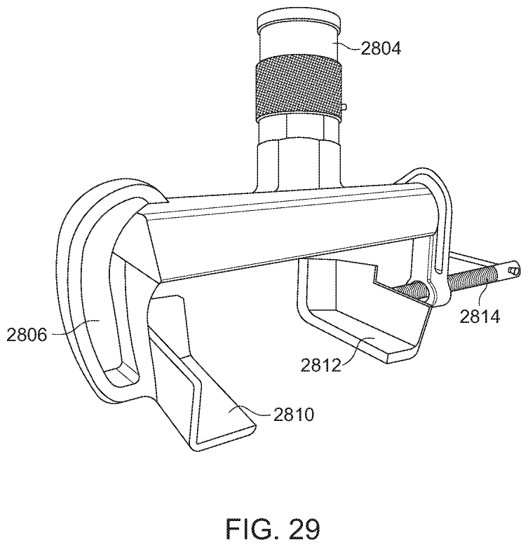

In another example, the tool interface can include a c-clamp. As illustrated in FIG. 28 and FIG. 29, a tool interface 2802 includes a coupling portion 2804 connected to a u-shaped bar or c-clamp 2806. At one side of the c-clamp 2806, a shoe 2810 is attached. At the opposite end of the c-clamp, a second shoe 2812 is connected to a threaded rod 2814 threaded through a portion of the c-clamp 2806. When the threaded rod 2814 is rotated, the shoe 2812 moves relative to the side of the c-clamp 2806, resulting in a clamping motion.

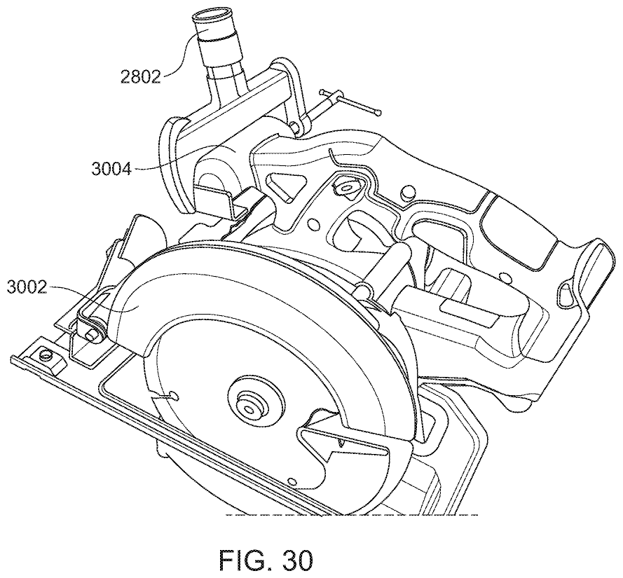

As illustrated in FIG. 30, the c-clamp interface 2802 can be secured to various tools having a bar interface. For example, a circular saw 3002 includes a circular bar interface 3004 to which the clamp 2802 can be secured.

In a first embodiment, a prosthetic device adaptor includes a tool interface, a coupling connected to the tool interface, and a prosthetic interface connected to the coupling.

In an example of the first embodiment, the prosthetic interface is to connect with a Hosmer-style hook.

In another example of the first embodiment and the above examples, the tool interface includes a plate and strap.

In a further example of the first embodiment and the above examples, the tool interface includes a clamp For example, the clamp is a circular clamp. In another example, the clamp is a c-clamp. For example, the c-clamp includes shoes to engage a tool handle.

In an additional example of the first embodiment and the above examples, the tool interface includes a threaded connector to directly engage a frame or casing of a tool.

In another example of the first embodiment and the above examples, the coupling is fixed.

In a further example of the first embodiment and the above examples, the coupling permits rotation around an axis. For example, the axis extends from the tool interface to the prosthetic interface.

In an additional example of the first embodiment and the above examples, the coupling includes a first portion connected to the tool interface and a second portion connected to the prosthetic interface. For example, the first and second portions of the coupling are detachable from each other. In an example, the first portion is a female coupling and the second portion is a male coupling. In another example, the first portion includes a magnet to attract the second portion. In a further example, the coupling includes ball bearings.

In another example of the first embodiment and the above examples, 17 the first portion is connected to the tool interface and the second portion is connected to the prosthetic interface.

In a further example of the first embodiment and the above examples, the prosthetic interface includes a post plate and a grip plate connected to the post plate. For example, the post plate includes a concave contour, the grip plate connected approximately perpendicular to the post plate extending from an interior surface of the concave contour. In another example, the grip plate includes a lip extending out of a plane of the grip plate at an edge disposed distal from the post plate. For example, the prosthetic device further includes a second lip extending out of the plane of the grip plate in a direction opposite the first lip. In an additional example, the prosthetic device further includes a loop connected to the grip plate. For example, the loop is disposed at an edge of the grip plate parallel to the post plate.

In a second embodiment, an adapted tool includes a tool and a prosthetic device adaptor. The prosthetic device includes a tool interface, a coupling connected to the tool interface, and a prosthetic interface connected to the coupling.

In an example of the second embodiment the tool includes a power tool. For example, the power tool includes a reciprocating saw, a drill, a power drill, a circular saw, a chainsaw, a jigsaw, a grinder, or a sander.

In another example of the second embodiment and the above examples, the tool includes a shovel, hoe, or rake.

In a further example of the second embodiment and the above examples, the prosthetic interface is to connect with a Hosmer-style hook.

In an additional example of the second embodiment and the above examples, the tool interface includes a plate and strap.

In another example of the second embodiment and the above examples, the tool interface includes a clamp For example, the clamp is a circular clamp. In another example, the clamp is a c-clamp. In a further example, the c-clamp includes shoes to engage a tool handle.

In a further example of the second embodiment and the above examples, the tool interface includes a threaded connector to directly engage a frame or casing of a tool.

In an additional example of the second embodiment and the above examples, the coupling is fixed.

In another example of the second embodiment and the above examples, the coupling permits rotation around an axis. For example, the axis extends from the tool interface to the prosthetic interface.

In a further example of the second embodiment and the above examples, the coupling includes a first portion connected to the tool interface and a second portion connected to the prosthetic interface. For example, the first and second portions of the coupling are detachable from each other. In an example, the first portion is a female coupling and the second portion is a male coupling. In another example, the first portion includes a magnet to attract the second portion. In a further example, the coupling includes ball bearings.

In an additional example of the second embodiment and the above examples, the first portion is connected to the tool interface and the second portion is connected to the prosthetic interface.

In another example of the second embodiment and the above examples, the prosthetic interface includes a post plate and a grip plate connected to the post plate. For example, the post plate includes a concave contour, the grip plate connected approximately perpendicular to the post plate extending from an interior surface of the concave contour. In another example, the grip plate includes a lip extending out of a plane of the grip plate at an edge disposed distal from the post plate. For example, the adapted tool further includes a second lip extending out of the plane of the grip plate in a direction opposite the first lip. In another example, the adapted tool further includes a loop connected to the grip plate. For example, the loop is disposed at an edge of the grip plate parallel to the post plate.

In a third embodiment, a method for preparing a tool includes securing a tool interface of a prosthetic adapter to the tool. The prosthetic adapter includes a tool interface, a coupling connected to the tool interface, and a prosthetic interface connected to the coupling. Examples of the third embodiment are found in the above examples of the prosthetic device and adapted tool.

In a fourth embodiment, a method for preparing a tool includes securing a tool interface of a prosthetic adapter to the tool. The prosthetic adapter includes a tool interface, a coupling including a first portion connected to the tool interface, and a prosthetic interface connected to a second portion of the coupling. The method further includes selecting the prosthetic interface configured for the tool and connecting the selected prosthetic interface to the tool interface. Examples of the fourth embodiment are found in the above examples of the prosthetic device and adapted tool.

In a fifth embodiment, a method for using an adapted tool includes selecting the adapted tool. The adapted tool includes a tool and a prosthetic adapter. The prosthetic adapter includes a tool interface, a coupling including a first portion connected to the tool interface, and a prosthetic interface connected to a second portion of the coupling. The method further includes engaging the prosthetic interface of the prosthetic adapter with a prosthetic. Examples of the fifth embodiment are found in the above examples of the prosthetic device and adapted tool.

It should be understood that various rotatable couplings can be utilized as alternatives to the illustrated rotatable coupling. Further, alternative tool interfaces can be envisaged based on the illustrated examples.

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” are employed to describe elements and components described herein. This is done merely for convenience and to give a general sense of the scope of the invention. This description should be read to include one or at least one and the singular also includes the plural unless it is obvious that it is meant otherwise.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

After reading the specification, skilled artisans will appreciate that certain features are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range.

1. A prosthetic device adaptor comprising:
a tool interface;
a coupling connected to the tool interface; and
a prosthetic interface connected to the coupling.

2. The prosthetic device adaptor of claim 1, wherein the prosthetic interface is to connect with a Hosmer-style hook.

3. The prosthetic device adaptor of claim 1, wherein the tool interface includes a plate and strap.

4. The prosthetic device adaptor of claim 1, wherein the tool interface includes a clamp.

5. The prosthetic device adaptor of claim 4, wherein the clamp is a circular clamp.

6. The prosthetic device adaptor of claim 4, wherein the clamp is a c-clamp.

7. The prosthetic device adaptor of claim 6, wherein the c-clamp includes shoes to engage a tool handle.

8. The prosthetic device adaptor of claim 1, wherein the tool interface includes a threaded connector to directly engage a frame or casing of a tool.

9. The prosthetic device adaptor of claim 1, wherein the coupling is fixed.

10. The prosthetic device adaptor of claim 1, wherein the coupling permits rotation around an axis.

11. The prosthetic device adaptor of claim 10, wherein the axis extends from the tool interface to the prosthetic interface.

12. The prosthetic device adaptor of claim 1, wherein the coupling includes a first portion connected to the tool interface and a second portion connected to the prosthetic interface.

13. The prosthetic device adaptor of claim 12, wherein the first and second portions of the coupling are detachable from each other.

14. The prosthetic device adaptor of claim 13, wherein the first portion is a female coupling and the second portion is a male coupling.

15. The prosthetic device adaptor of claim 13, wherein the first portion includes a magnet to attract the second portion.

16. The prosthetic device adaptor of claim 13, wherein the coupling includes ball bearings.

17. The prosthetic device adaptor of claim 1, wherein the first portion is connected to the tool interface and the second portion is connected to the prosthetic interface.

18. The prosthetic device adaptor of claim 1, wherein the prosthetic interface includes a post plate and a grip plate connected to the post plate.

19. The prosthetic device adaptor of claim 18, wherein the post plate includes a concave contour, the grip plate connected approximately perpendicular to the post plate extending from an interior surface of the concave contour.

20. The prosthetic device adaptor of claim 18, wherein the grip plate includes a lip extending out of a plane of the grip plate at an edge disposed distal from the post plate.

21. The prosthetic device adaptor of claim 20, further comprising a second lip extending out of the plane of the grip plate in a direction opposite the first lip.

22. The prosthetic device adaptor of claim 18, further comprising a loop connected to the grip plate.

23. The prosthetic device adaptor of claim 22, wherein the loop is disposed at an edge of the grip plate parallel to the post plate.

24.-52. (Canceled)
